(19) United States
(12) Patent Application Publication
(10) Pub. No.: US 2016/0101137 A1
(43) Pub. Date: Apr. 14, 2016

(54) MICROORGANISM BIOMASS FOR PREVENTION AND REDUCTION OF THE ADVERSE EFFECTS OF PATHOGENS IN DIGESTIVE TRACT

(71) Applicant: NESTE OIL CORPORATION, Espoo (FI)

(72) Inventors: Jukka-Pekka PASANEN, Helsinki (FI); Juha APAJALAHTI, Helsinki (FI); Eero PENNALA, Espoo (FI)

(21) Appl. No.: 14/893,760

(22) PCT Filed: May 26, 2014
(86) PCT No.: PCT/EP2014/060860

§ 371 Date: Nov. 24, 2015

(30) Foreign Application Priority Data
May 24, 2013 (EP) 13169245.1

(51) Int. Cl. A61K 36/062; A01N 63/04

(52) U.S. Cl. CPC A61K 36/062; A01N 63/04; A23K 1/007; A23K 1/008; A23K 1/188; A23K 1/1826; A23K 1/184; A23L 1/03; A23V 2002/00

(57) ABSTRACT

The present invention relates to microorganism biomass and a feed or food composition which can be used in preventing or reducing the adverse effects of pathogens in animal or human digestive tract. The biomass comprises non-living microorganism biomass obtainable by cultivating microorganism strains on a cultivation medium comprising lignocellulosic material.

### FIELD OF THE INVENTION

The present invention relates to microorganism biomass, or a feed or food composition comprising said biomass for use in preventing or reducing the adverse effects of pathogens in animal or human digestive tract. The invention relates also to a feed or food composition and to a method for producing it. The present invention relates also to a method for improving the well-being and increasing the productivity of an animal.

### BACKGROUND OF THE INVENTION

The digestive tract of human and animals may be infected by many pathogens. In human this causes disorders in digestive tract or illnesses, in animals this causes economic losses due to lowered productivity, illnesses or death. In addition, pathogens may cause food borne infections for consumers, if entered into the food chain. Many of the pathogens are part of the normal intestinal flora, and illness may be triggered for example by stress.

Pathogenic Salmonella enterica strains are a distressing health problem both with humans and animals worldwide. S. enterica serotype Choleraesuis shows the highest tendency to cause systemic infections. Emergence of S. enterica strains that are resistant to chloramphenicol and other antibiotics has aroused concern about the use of these agents for the treatment of systemic infection caused by Salmonella. Salmonella enterica is a facultatively anaerobic Gram-negative bacterium.

Escherichia coli is also part of the normal microbiota of animal or humans. Some E. coli serotypes are harmful, e.g. E. coli F4+. E. coli are the major cause of weaning diarrhea in piglets. Escherichia coli is a Gram-negative bacterium.

Kogan and Kocher (2007. Role of yeast polysaccharides in pig nutrition and health protection, Livestock science, 109 (2007)161-165) describe that certain yeast cell wall polysaccharides were able to block fimbriae of pathogenic bacteria and thus prevent their adhesion to the mucous epithelium. The bacterial lectin binding capacity of one commercial yeast wall polysaccharide preparation Bio-Mos® (Alltech, Inc.) was tested with agglutination tests with 258 pathogenic strains of four bacterial genera.

Additionally, the binding of pathogens has been reported by using different adsorbents, for example activated charcoal (Knutson et al. 2006. Effects of activated charcoal on binding E. coli O157:H7 and Salmonella typhimurium in sheep, Small Ruminant Res. 65 (2006) 101-105).

US 201210034344A1 (Menon et al) describes a method for producing lipids from lignocellulosic feedstock by fermentation. In the method, bacterial and/or fungal strains, such as Trichoderma reesei, were used to convert cellulose, hemicellulose or glycerol into lipids and fungus of the genus Penicillium was used to degrade cellulose into sugars, which could be used by the lipid producing microbes. In the method described in the publication fungal biomass depleted of a liquid product was used for animal meal.

Although some attempts to eliminate or reduce the effects of pathogens in digestive tract are known from the prior art, there is still a need for efficient products and methods which could be used against pathogens in the digestive tract of human and animals.

### SUMMARY OF THE INVENTION

One object of the present invention is to provide a solution to the problems encountered in the prior art. Specifically, the present invention aims to provide a solution to problems encountered in the well-being of human and animals. Furthermore, the present invention aims to increase the productivity of animals.

In particular, it is one object of the present invention to provide a solution, which enables prevention or reduction of the adverse effects of pathogens in animal or human digestive tract.

To achieve these objects the invention is characterized by the features that are enlisted in the independent claims. Other claims represent the preferred embodiments of the invention.

The invention is based on the finding that microorganism biomass prevents and reduces the adverse effects of pathogens in animal or human digestive tract.

It has now been surprisingly found that microorganism biomass, in particular biomass, which has been obtained by cultivating microorganisms on a cultivation medium comprising lignocellulosic material has this ability of preventing and reducing the adverse effects of pathogens.

Hence, in one aspect, the present invention provides microorganism biomass for use in preventing or reducing the adverse effects of pathogens in animal or human digestive tract as defined in claim 1.

In another aspect, the present invention provides a feed or food composition for use in preventing or reducing the adverse effects of pathogens in animal or human digestive tract as defined in claim 11.

In a further aspect, the present invention provides a feed or food composition as defined in claim 16.

In a still further aspect, the present invention provides a method for improving the well-being and increasing the productivity of an animal as defined in claim 18.

In a still further aspect, the present invention provides a method for producing a feed or food composition.

In still further aspects, the present invention provides a method, a microorganism biomass, and a feed or food composition for preventing or reducing the adverse effects of pathogens in animal or human digestive tract.

### BRIEF DESCRIPTION OF THE FIGURES

FIGS. 1 and 3 show the effect of fungal biomass on the binding of pathogen E. coli F4+(K88) on piglet intestinal epithelium.

FIGS. 2 and 4 show the effect of fungal biomass on the binding of S. enterica serovar Dublin on piglet intestinal epithelium.

### DETAILED DESCRIPTION OF THE INVENTION

### Definitions

The present invention provides microorganism biomass for use in preventing or reducing the adverse effects of pathogens in animal or human digestive tract. Such biomass is obtainable by cultivating said microorganisms on a cultivation medium comprising lignocellulosic material.

According to this disclosure microorganism biomass obtainable by cultivating microorganism strains in a cultivation medium comprising lignocellulosic material, typically lignocellulose hydrolysate or saccharides has been shown to inhibit the binding of pathogens originating from contaminated feed.

By “lignocellulosic material” is meant any material which comprises lignocellulose. Lignocellulosic material comprises lignocellulose, fragments of lignocellulose or hydrolysis products of lignocellulose including saccharides, such as mono-, di-, and/or oligosaccharides originating from lignocellulose. “Lignocellulosic material” may comprise also other components in addition to lignocellulose such as other components from wood or herbaceous plants.

“Lignocellulose” comprises carbohydrate polymers cellulose and hemicellulose and aromatic polymer lignin. Lignocellulosic material include but is not limited to woody plants or non-woody, herbaceous plants or other materials containing cellulose and/or hemicellulose: Materials can be agricultural residues (such as wheat straw, rice straw, chaff, hulls, corn stover, sugarcane, bagasse), dedicated energy crops (such as switchgrass, Miscanthus, reed canary grass, willow, water hyacinth), wood materials or residues (including sawmill and pulp and/or paper mill residues or fractions, such as hemicellulose, spent sulphite liquor, waste fibre and/or primary sludge), moss or peat, or municipal paper waste. The term lignocellulosic material comprises also low lignin materials, materials such as macroalgae biomass. In addition, the materials comprise also hemicellulose or cellulose fractions from industrial practices. The term lignocellulosic material encompasses any kind of cellulose fraction. The raw materials or certain fractions, such as hemicellulose and/or cellulose, of raw materials from different origin, plant species, or industrial processes can be mixed together and used as raw materials for cultivating microorganism biomass according to this disclosure.

The term lignocellulosic material comprises at least 50 wt % lignocellulose, preferably at least 60 wt % lignocellulose, more preferably at least 70 wt % lignocellulose, most preferably at least 80 wt % lignocellulose. Usually lignocellulosic material comprises 60-95 wt % lignocellulose, typically 70-90 wt % or 80-90 wt % lignocellulose.

“A cultivation medium comprising lignocellulosic material” means a cultivation medium for cultivating a microorganism, which medium comprises lignocellulose, fragments of lignocellulose or hydrolysis products of lignocellulose including, nitrogen compounds originating from proteins and metals, and other components necessary for cultivating said microorganism, such as a source of nitrogen, phosphorus, inorganic salts and/or trace elements. Lignocellulose may function as a carbon source for said microorganism, but it may also have other functions in the cultivation medium.

“Hydrolysis” refers here to saccharification of polymeric sugars to sugar oligomers and monomers. Saccharification is typically carried out in two phases: first the substrate i.e. lignocellulosic material or lignocellulose is hydrolyzed by thermochemical or chemical methods and then by using enzymes capable of hydrolysing polymeric sugars. Alternatively and depending on the lignocellulosic material saccharification can be carried out by using thermochemical or chemical methods or by enzymes capable of hydrolysing polymeric sugars or some combination of these methods. Chemical methods include, but are not limited to acid treatment.

In some embodiments of the invention, the microorganism cultivated on a medium comprising lignocellulosic material is able to hydrolyse lignocellulose to sugar oligomers and monomers. In other preferred embodiments lignocellulosic material or feedstock comprising polymeric sugars is hydrolyzed to monomeric sugars thermochemically and/or chemically and/or by enzymes before cultivation. In preferred embodiments of the invention, lignocellulosic material containing polymeric sugars is hydrolysed thermochemically and/or chemically and/or by enzymes to contain oligomeric sugars and the microorganism cultivated on a medium comprising lignocellulose is able to utilize these sugar oligomers.

By “lignocellulose hydrolysate” is meant the hydrolysis products of lignocellulose or lignocellulosic material comprising cellulose and/or hemicellulose, oligosaccharides, mono- and/or disaccharides, acetic acid, formic acid, other organic acids, furfural, hydroxymethyl furfural, levulinic acid, phenolic compounds, other hydrolysis and/or degradation products formed from lignin, cellulose, hemicellulose and/or other components of lignocellulose, nitrogen compounds originating from proteins, metals and/or non-hydrolyzed or partly hydrolyzed fragments of lignocellulose.

According to this disclosure the treatment of lignocellulose and production of lignocellulose hydrolysate for cultivation of microbial biomass can be done with any method known in the art or developed in the future. Methods include but are not limited to thermochemical treatment, steam explosion, hot water extraction, autohydrolysis, sub critical water treatment, super critical water treatment, strong acid treatment, mild acid treatment, alkaline treatment (e.g. lime, ammonia), Organosolv treatment (e.g. alcohols, organic acids), mechanical treatment, thermomechanical treatment, and ionic liquid treatment. These treatments methods can be combined with enzymatic treatment.

“A cultivation medium comprising saccharides” means here a cultivation medium, which comprises mono-, di-, and/or oligosaccharides from lignocellulose.

“A cultivation medium comprising pure saccharides” means here a cultivation medium, which comprises mono-, di- and/or oligosaccharides, which are not produced by hydrolysis of lignocellulose. Pure saccharides include, e.g. starch or starch derived sugars, sugar cane or sugar beet derived sugars.

In some embodiments of the invention, a microorganism is preferably cultivated on a medium comprising lignocellulose hydrolysate, in other embodiments preferably on a medium comprising mono-, di-, and/or oligosaccharides originating from lignocellulose or on pure saccharides. Depending on the pathogen either the whole range of hydrolysis products of lignocellulose, or saccharides (mono-, di- and/or oligosaccharides) obtained as hydrolysis products from lignocellulose or pure saccharides, give more effective biomass for preventing or reducing the adverse effects of said pathogen.

By “a microorganism” is meant any microorganism, typically a fungus, preferably a filamentous fungus or yeast, a heterotrophic algae, a bacterium or an archaebacterium, which microorganism is capable of producing cellular biomass. Microorganism is able to produce microbial biomass when grown on cultivation medium comprising lignocellulosic material. Preferably said microorganism is a filamentous fungus or yeast. Preferably said microorganism is a filamentous fungus or yeast. More preferably the microorganism biomass is fungal biomass, still more preferably obtainable from cultivation of filamentous fungi or yeasts, most preferably filamentous fungi from genus Aspergillus and/or Mortierella or yeasts from genus Rhodosporidium and/or Lipomyces.

“Cell mass” or “Microbial biomass” or “microorganism biomass” are used here synonymously and stand for a solid, semi-solid or flowing material fraction, which contains microorganisms or is treated for the recovery of specific products produced by said microorganism. Microorganism biomass comprises microorganism cells or residues of microorganism cells.

Typically microorganism biomass according to this disclosure means biomass of non-living microorganisms. Methods used for the recovery of co-products and/or microorganism biomass typically comprise treatments which destroy the structure of the microorganism cell, such as disrupt microbial cell wall. Microorganism biomass thus means in particular non-living microorganisms or their residues. According to this disclosure a microorganism can be cultivated for biomass production or it can be cultivated under conditions that permit the microorganism to produce desired products, such as lipids, or other economically valuable co-products. Typically the microorganism biomass according to this disclosure is obtained after recovery of lipids from microbial biomass.

Preferably, by “a microorganism” is meant here a lipid-producing, in another word oleaginous, microorganism. When a lipid-producing microorganism has been used for single cell oil production, the microorganism biomass is residual biomass from a single cell oil production process.

By “lipid producing microorganism” or “oleaginous microorganism” is meant a microorganism that is able to produce and accumulate in their cell biomass more than 15% lipids from their dry cell biomass weight when cultivated in suitable conditions for lipid production. Alternatively or in addition microorganism may be able to excrete lipids outside the cells e.g. to cultivation medium.

Single-cell oil stands typically for an intracellular lipid that has been intracellularly synthesized by a microorganism, lipids excreted by the cell, as well as lipids present in the structural parts of a cell, such as in membrane systems.

By “single-cell oil production process” is meant a process where microorganisms are used to produce oils. In the process, microorganisms are cultivated on organic carbon sources and microorganisms are let to produce oil. Microorganisms can store the oil intracellularly or excrete it out from the cell. Organic carbon source can be lignocellulosic material. Single-cell oil process typically utilizes microorganisms, such as oleaginous microorganisms, that are capable of producing lipids efficiently.

“Lipid recovery” or “oil recovery” refers to a process, in which the lipid (intracellular lipid) is recovered by mechanical, chemical, biochemical, thermomechanical or autocatalytic methods or by a combination of these methods from the microorganism cells.

After cultivation of microorganisms for production of oil, microorganisms containing lipids may be separated from culture medium by any known methods, such as by using a filtration or decanting techniques. Alternatively, centrifugation with industrial scale commercial centrifuges of large volume capacity may be used to separate the desired products.

In various embodiments of the invention, oil, or precursors for oil, may be recovered from cell biomass or culture broth using any method known in the art or developed in the future. Such methods, include, but are not limited to extraction with organic solvents or mechanical pressing. In various embodiments of the invention, microorganism cells may be disrupted to facilitate the separation of oil and other components. Any method known for cell disruption may be used, such as ultrasonication, osmotic shock, mechanical shear force, cold press, thermal shock, enzyme-catalyzed or self-directed autolysis.

“Residual cell mass” or “residual microbial biomass” or “residual microorganism biomass” are used here synonymously and stand for a solid, semi-solid or flowing material fraction, which contains microorganisms treated for the recovery of intracellular lipids and/or other products.

The term “lipid” refers to a fatty substance, whose molecule generally contains, as a part, an aliphatic hydrocarbon chain, which dissolves in nonpolar organic solvents but is poorly soluble in water. Lipids are an essential group of large molecules in living cells. Lipids are, for example, fats, oils, waxes, wax esters, sterols, terpenoids, isoprenoids, carotenoids, polyhydroxyalkanoates, fatty acids, fatty alcohols, fatty acid esters, phospholipids, glycolipids, sphingolipids and acylglycerols. The term “lipid” and “oil” are used in this description synonymously. The term “acyglycerol” refers to an ester of glycerol and fatty acids. Acylglycerols occur naturally as fats and fatty oils. Examples of acylglycerols include triacylglycerols (TAGs, triglycerides), diacylglycerols (diglycerides) and monoacylglycerols (monoglycerides).

In embodiments where microorganism biomass is cultivated on lignocellulosic material, in particular lignocellulose hydrolysate, it may comprise also hydrolysis products or residues of lignocellulose. Such residues are for example, acetic acid, formic acid, other organic acids, furfural, hydroxymethyl furfural, levulinic acid, phenolic compounds, other hydrolysis or degradation products formed from lignin, cellulose or hemicellulose or other components of lignocellulose, or non-hydrolyzed or partly hydrolyzed fragments of lignocellulose. Hence, if Microorganism biomass is cultivated on lignocellulose hydrolysate, it typically comprises lignocellulose hydrolysis products or residues, which the microorganism has not utilized during cultivation or which the microorganism has not been able to utilize, for example lignin, polysaccharides and mono-, dior oligosaccharides. The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass. Depending on the source of the lignocellulosic material and on other components of the cultivation medium, “microorganism biomass” may comprise for example residues of various proteins and lipids.

In preferred embodiments of the invention, the microorganism is cultivated on lignocellulosic hydrolysate containing lignocellulose hydrolysis or degradation products not utilized by microorganism, such as phenolic compounds and furfural. These compounds are formed in the hydrolysis of lignocellulose. The lignocellulose hydrolysis can be performed by thermochemical treatment, steam explosion, hot water extraction, autohydrolysis, sub critical water treatment, super critical water treatment, strong acid treatment, mild acid treatment, alkaline treatment (e.g. lime, ammonia), Organosolv treatment (e.g. alcohols, organic acids), mechanical treatment, thermomechanical treatment, ionic liquid treatment in addition to enzymatic treatment.

Without being bound by any theory it seems that lignocellulosic residues in microorganism biomass may be at least partly responsible for the prevention or reduction of adverse effect of pathogens in digestive tract. The components of hydrolysate, such as phenolic compounds and metals may adsorb to the cell wall of microorganisms and be carried on with the microbial biomass. Alternatively, the components of the lignocellulose hydrolysate may occupy the pathogen binding sites on intestinal mucus, thus preventing their adherence. Alternatively, microbial cell walls may be modified by the components of lignocellulose hydrolysate.

The capability of microorganism biomass according to this disclosure to prevent or reduce the adverse effect of pathogens may also be due to process driven changes in rheology of the biomass and/or changes in the cell wall composition of the biomass. These changes in biomass rheology and composition of the cell wall can potentially be due to single cell production process and/or due to the use of lignocellulosic hydrolysates in the cultivation.

Single cell oil production process typically uses nutrient starvation, such as nitrogen or phosphorus starvation to allow microorganisms to produce and accumulate oil. The starvation in single cell production process can result in changes in biomass rheology and composition of the cell wall.

Preferred microorganism strains for the purposes of the present invention are from the species and genera listed below:

Preferred fungal strains are from species from genera Aspergillus such as Aspergillus oryzae, Mortierella such as Mortierella isabellina, Chaetomium, Claviceps, Cladosporidium, Cunninghamella, Emericella, Fusarium, Glomus, Mucor, Paecilomyces, Penicillium, Pseudozyma, Pythium, Rhizopus, Tremella, Trichoderma, Zygorhynchus, Humicola, Cladosporium, Malbranchea, Umbelopsis such as Umbelopsis isabellina and Ustilago. Most preferred fungal species are from genera Aspergillus and/or Mortierella. Preferred fungi are those fungi capable of producing effectively lipids.

Preferred yeast strains are those belonging to species from genera Clavispora, Geotrichum, Deparyomyces, Pachysolen, Kluyveromyces, Galactomyces, Hansenula, Leucosporidium, Saccharomyces, Sporobolomyces, Sporidiobolus, Waltomyces, Endomycopsis, Cryptococcus, such as Cryptococcus curvatus, Rhodosporidium, such as Rohodosporidium toruloides, Rhodotorula, such as Rhodotorula glutinis, Yarrowia, such as Yarrowia lipolytica, Pichia, such as Pichia stipitis, Candida such as Candida curvata, Lipomyces such as Lipomyces starkeyi and Trichosporon such as Trichosporon cutaneum or Trichosporon pullulans. Preferred yeasts are those yeasts capable of producing effectively lipids.

Preferred bacteria are those belonging to the species from genera Acinetobacter, Actinobacter, Alcanivorax, Aerogenes, Anabaena, Arthrobacter, Bacillus, Clostridium, Dietzia, Gordonia, Escherichia, Flexibacterium, Micrococcus, Mycobacterium, Nocardia, Nostoc, Oscillatoria, Pseudomonas, Rhodococcus, Rhodomicrobium, Rhodopseudomonas, Shewanella, Shigella, Streptomyces and Vibrio. Preferred bacteria are those bacteria capable of producing effectively lipids.

Most preferred algae are microalgae, such as microalgae species from genera comprising Achnantes, Amphiprora, Amphora, Ankistrodesmus, Attheya, Boeklovia, Botryococcus, Biddulphia, Brachiomonas, Bracteococcus, Carteria, Chaetoceros, Characium, Chlamydomonas, Crypthecodinium, Cryptomonas, Chlorella, Chlorococcum, Chrysophaera, Coccochloris, Cocconeis, Cyclotella, Cylindrotheca, Dunaliella, Ellipsoidon, Entomoneis, Euglena, Eremosphaera, Extubocellulus, Franceia, Fragilaria, Gleothamnion, Hantzschia, Haematococcus, Hormotilopsis, Hymenomonas, lsochrysis, Lepocinclis, Melosira, Minidiscus, Micractinum, Monallanthus, Monoraphidium, Muriellopsis, Nannochloris, Nannochloropsis, Navicula, Neochloris, Nephroselmis, Nitzschia Ochromonas, Oedogonium, Oocystis, Papiliocellulus, Parachlorella, Pascheria, Pavlova, Peridinium, Phaeodactylum, Plankthothrix, Platymonas, Pleurochrysis, Pleurosigma, Porphyridium, Prototheca, Prymnesium, Pseudochlorella, Pyramimonas, Pyrobotrus, Radiosphaera, Rhodomonas, Rhodosorus, Sarcinoid, Scenedesmus, Schizochytrium, Scrippsiella, Seminavis, Skeletonema, Spirogyra, Stichococcus, Synedra, Tetraedron, Tetraselmis, Thalassiosira, Trachyneis, Traustrochytrium, Trentepholia, Ulkenia, Viridiella, and Volvox. Preferred microalgae are those microalgae capable of growing heterotrophically and producing effectively lipids. The organisms belonging to the genera Schizochytrium, Thraustochytrium and Crypthecodinium and Ulkenia are sometimes called as marine fungi.

According to the present disclosure microorganism biomass obtainable by cultivating microorganisms on a cultivation medium comprising lignocellulosic material prevents or reduces the adverse effects of pathogens in animal or human digestive tract, preferably in animal or human stomach, small intestine and/or colon.

Preferably said biomass prevents or reduces the adverse effects of pathogens. By “pathogen” is hear meant any microorganism, typically a bacterium or a fungus that causes disease or disorder in animal or human.

Pathogens comprise species for example from genera Salmonella, Shigella, Campylobacter, Clostridium, e.g. C. difficile or C. perfringens, Vibrio, e.g. V. cholera, V. parahaemolyticus, Staphylococcus aureus and Escherichia coli. Preferably said biomass prevents or reduces the adverse effects of pathogens Escherichia coli and/or Salmonella enterica.

By “preventing or reducing the adverse effects of pathogens” is meant here the increase in well-being and productivity of animals, the productivity of said animals being decreased by animal diseases or disorders caused by pathogens, or high mortality of said animals being caused by pathogens. In humans preventing or reducing the adverse effects of pathogens means the increase in well-being and better health.

The present invention provides also a feed or food composition for use in preventing or reducing the adverse effects of pathogens in animal or human digestive tract. Said feed or food composition comprises the microorganism biomass according to this disclosure.

The feed or food composition comprises microorganism biomass preferably 0.001-20 wt %, more preferably 0.1-3 wt % dry weight of dry weight of said composition.

In some embodiments of the invention the composition is a ruminant or monogastric animal, such as pigs or poultry feed composition. The composition comprises microorganism biomass and components or ingredients suitable for ruminant or monogastric animal feed composition, such as source of protein and fibre components.

In some other embodiments of the invention the composition is a fish feed composition. The composition comprises microorganism biomass and components or ingredients suitable for fish feed composition, such as source of protein and fat components.

In further embodiments of the invention the food composition is a nutritional product for human use. The composition comprises microorganism biomass and components or ingredients suitable for food composition. Such components comprise for example salt and sugar or other flavour giving ingredients.

By “feed or food ingredient” is here meant a component part or constituent of any combination or mixture making up a feed or food, whether or not it has a nutritional value in the animal's or human's diet, including feed or food additives. Ingredients are of plant, animal or aquatic origin, or other organic or inorganic substances.

The feed or food composition for use in preventing or reducing the adverse effects of pathogens in animal or human digestive tract may be used in the composition as a food or feed topping, food or feed extender or feed or food supplement.

The present invention provides also a feed or food composition, which comprises preferably 0.001-20 wt %, more preferably 0.1-3 wt % dry weight of dry weight of said composition non-living microorganism biomass, said microorganism biomass being obtainable as disclosed herein, and preventing or reducing the adverse effects of pathogens in animal or human digestive tract.

The present invention provides also a method for producing a feed or food composition. Preferable the method comprises the steps of
cultivating a microorganism on a cultivation medium comprising lignocellulosic material;
collecting microorganism cells from said cultivation medium;
optionally rupturing or lysing the cultured cells;
separating the solid phase containing microbial biomass and/or separating the cell wall from the soluble intracellular components and obtaining a cell wall extract and optionally extracting said biomass or cell extract by organic acid, typically hexane;
drying and optionally desolventizing the separated residual microbial biomass or cell extract; and
adding said cell mass or cell extract to feed or food.

The present invention provides also a method for producing a feed or food composition from single cell oil production process. Preferable the method comprises the steps of
cultivating a microorganism on a cultivation medium comprising lignocellulosic material;
allowing microorganisms to produce oil, preferably under nutrient starvation;
collecting oil-rich microorganism cells from cultivation medium and optionally drying the cells;
rupturing or lysing the cultured cells;
recovering oil from microorganisms cells typically by solvent, e.g. hexane, said extraction creating liquid phase containing oil and residual microorganism biomass,
separating the residual microorganism biomass;
drying the separated residual microorganism biomass; and
adding said microorganism biomass to feed or food.

Separated residual microorganism biomass may be treated mechanically, thermochemically, chemically or enzymatically prior to adding to feed or food.

Advantageously a feed or food composition is prepared by mixing a specific amount of microorganism biomass or cell wall extract to feed or food. A feed or food composition is prepared to comprise preferably 0.001-20 wt %, more preferably 0.1-3 wt % (dry weight) of dry weight of said composition microorganism biomass obtainable as disclosed herein.

Advantageously the microorganism biomass comprises residues of lignocellulose. The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass.

The present invention provides also a method for improving the well-being and increasing the productivity of an animal. The method comprises feeding to said animal a feed composition comprising non-living microorganism biomass, said biomass being obtainable by cultivating a microorganism on a cultivation medium comprising lignocellulosic material as disclosed herein.

Preferably the composition comprises 0.001-20 wt %, more preferably 0.1-3 wt % dry weight of dry weight of said composition microorganism biomass.

Preferably the microorganism biomass is microorganism biomass as disclosed here earlier. Advantageously the microorgaism biomass comprises residues of lignocellulose The amount of lignocellulose hydrolysis products or residues is typically 0.01-20 wt % (dry weight), usually 0.5-10 wt % of dry weight of the microorganism biomass. The amount of phenolic compounds originating from lignocellulosic materials is typically 0.01-10 wt % (dry weight), usually 0.05-5 wt % of dry weight of the microorganism biomass.

In some embodiments of the invention the composition is a ruminant or monogastric animal feed composition.

In some other embodiments of the invention the composition is a fish feed composition.

In further embodiments of the invention the food composition is a nutritional product for human use.

The microorganism biomass may be used in the feed or food composition as a feed or food topping, feed or food extender or feed or food supplement.

According to this disclosure the microorganism biomass obtainable by cultivating microorganisms on a medium comprising lignocellulosic material is used to prevent or reduce the adverse effects of pathogens in animal or human digestive tract. The prevention or reduction effect is proposed to be based on the ability of the biomass to inhibit the adherence of pathogens in animal or human digestive tract and thereby prevent pathogens from entering the rest of the body. Inhibition of pathogens binding to the intestinal epithelium can be due to efficient co-flocculation of the pathogenic bacteria with insoluble part of the microorganism biomass, which prevents bacterial binding and leads to washout of the agglomerates. The other possible mechanism is that the microorganism biomass masks the adhesins on the surface of the pathogenic bacteria or receptors on the surface of the intestinal epithelium, thus preventing the adherence.

The present invention has been exemplified by showing that microorganism biomass obtainable from cultivation of Aspergillus, Mortierella, and Rhodosporidium with pure saccharides or with lignocellulosic hydrolysate can be used as a binding agent for E. coli and S. enterica in the animal digestive tract. The results indicate that depending on the pathogen either the microorganism biomass obtained from cultivation with lignocellulosic hydrolysate or with saccharides performs better as a binding agent.

The microorganism biomasses from microorganisms grown on lignocellulosic hydrolysates inhibit also the binding of other pathogens to intestinal epithelium than those described in the Examples and reduce the adverse effects by other pathogens than those described in the Examples to animals or humans.

Microorganism biomass from Aspergillus, Mortierella and Rhodosporidium grown on pure saccharides have similar inhibition patterns for both E. coli and S. enterica.

Microorganism biomass from Aspergillus and Rhodosporidium grown on lignocellulose hydrolysate seemed to specifically inhibit E. coli binding even at low dose (similar to yeast cell wall product used as a positive control). The used dose was 0.1 mg/ml or higher.

The effective components in yeast are unknown, but it is hypothesized that carbohydrate components in yeast mask pathogen specific binding receptors on the intestinal epithelium.

Microorganism biomass from Aspergillus, Mortierella, and Rhodosporidium grown on pure saccharides inhibited dose dependently both E. coli and S. enterica adherence. This effect was of similar magnitude to that of the positive control.

Microorganism biomass from Aspergillus and Rhodosporidium grown on lignocellulose hydrolysates inhibited E. coli adherence by about 50% at all four doses. S. enterica inhibition was detected with the highest dose levels used.

The invention will hereafter be described by way of the following non-limiting items.

### Items

Item 1. A method for preparing a feed additive, said method comprising the steps of
(a) cultivating one or more microorganism(s) in a cultivation medium comprising lignocellulosic material,
(b) subject said microorganism(s) to a step of cell disruption to obtain non-living biomass of said microorganism(s)
(c) isolate a solid phase containing the microbial biomass and residues of lignocellulose of step (b),
(d) optionally, drying the microbial biomass obtained from step (c).

Item 2. The method of item 1, wherein the microbial biomass obtained from (c) is subjected to washing step.

Item 3. The method according to any of the preceding items, wherein said cultivation medium comprising lignocellulose hydrolysate.

Item 4. The method of according to any of the preceding items, wherein said cultivation medium comprises starch and/or sugar cane/beet derived sugars.

Item 5. The method of according to any of the preceding items, wherein the microbial biomass is obtained from oleaginous microorganisms.

Item 6. The method of according to any of the preceding items, wherein said microbial biomass is residual microbial biomass obtained from a fermentation process.

Item 7 The method of according to item 5, wherein the microbial biomass of step (c) is obtained by subjecting the oleaginous microorganisms to a step of removing the microbial oil from said microbial biomass.

Item 8. The method of according to item 7, wherein the microbial biomass of step (c) is obtained by subjecting the oleaginous microorganisms to a step of removing the microbial oil from said microbial biomass after step b).

Item 9. The method of according to any of the preceding items, wherein the microbial biomass is obtained from one or more microorganism.

Item 10. The method of according to any of the preceding items, wherein said microbial biomass is obtained from one or more fungi, preferably yeasts or filamentous fungi.

Item 11. The method of according to any of the preceding items, wherein said microbial biomass is obtained from Rhodosporidium.

Item 12. The method of according to any of the preceding items, wherein said microbial biomass is obtained from Aspergillus or Mortierella.

Item 13. A feed or food additive obtainable from the method of any one of items 1-12.

Item 14. A method for inactivating a pathogen in a contaminated feed or food product, said method comprising the steps of
(a) providing a feed or food product contaminated with a pathogen,
(b) providing a feed or food additive according to item 13,
(c) mixing the feed or food product of (a) with the feed or food additive of (b).

Item 15. A method for preparing a feed or food product, said method comprising the steps of
(a) providing a feed or food substance,
(b) providing a feed or food additive according to item 13,
(c) mixing the feed or food substance of (a) with the feed or food additive of (b) to obtain a feed or food product.

Item 16. The method according to any of items 14 or 15, wherein the feed additive amounts 0.001-20 wt % dry weight of the feed of step (c).

Item 17. The method according to any of items 14 to 16, wherein the feed additive amounts 0.1-3 wt % dry weight of the feed of step (c).

Item 18. The method according to any of items 14 to 17, wherein the feed or food product is contaminated with a pathogen selected from the list consisting of Salmonella genera and E. coli or a combination of said pathogens.

Item 19. The method according to any of items 14 to 18, wherein the feed or food product is contaminated with a pathogen selected from the list consisting of Escherichia coli and Salmonella enterica or a combination of said pathogens.

Item 20. A feed or food product obtainable from any one of items 15 to 19.

Item 21. A method for feeding an animal, said method comprising
(a) feeding said animal with a feed product according to item 20.

Item 22. The method according to item 21, wherein said animal is a ruminant or monogastric animal.

Item 23. The method according to item 21, wherein said animal is aquaculture.

Item 24. The method according to any of items 21 to 23, wherein the animal is a livestock.

Item 25. The method according to item 21, wherein said animal is a livestock selected from the list consisting of pigs, horses, poultry, cattle, goats and sheep.

Item 26. The method according to item 21, wherein said animal is a chicken.

Item 27. The method according to item 21, wherein said animal is a pig

The invention is illustrated by the following non-limiting examples. The invention can be applicable to other pathogens than those illustrated in examples. The invention can be applicable to biomasses from other microorganisms than those illustrated in the examples. It should be understood, however, that the embodiments given in the description above and in the examples are for illustrative purposes only, and that various changes and modifications are possible within the scope of invention.

### EXAMPLES

### Example 1

### Preparation of Aspergillus Biomass Grown on Lignocellulose Hydrolysate; Batch 1

Hemicellulose hydrolysate was prepared from pelletized wheat straw by batch hot water extraction. Temperature of the extraction was 180° C. and the extraction time 1 hour. Solid material was removed from the hydrolysate by filtration. After this the hydrolysate was evaporated. The evaporated hydrolysate had 4.7 wt-% of phenolics in the whole dry matter content.

Filamentous fungus Aspergillus oryzae, strain DSM 1864 (or other A. oryzae strain which are readily available from recognized microbial culture collections) was grown under aeration in a 5-liter fermentor. First 26 hours of the fermentation were done as a batch fermentation using sucrose. After this the fermentation was done as a continuous fermentation and hemicellulose medium (lignocellulose hydrolysate from which solid material had been removed as described above) was added continuously to the fermentor. Flow rate of the medium was 0.1 l/h and total cultivation time 98 h. Growth media was supplemented with yeast extract (5 g/l), (NH4)2SO4 (1.5 g/l), MgSO4 (1 g/l), KH2PO4 (1 g/l), K2HPO4 (2 g/l) and CaCl2 (0.1 g/l).

After cultivation biomass was inactivated by heat, harvested by filtration, washed using tap-water and freeze-dried. Dried biomass was pulverized by milling and oil extracted using n-heptane. Residual solvent was removed by drying the biomass by efficient ventilation.

The dried biomass was used in pathogen binding tests.

### Preparation of Aspergillus Biomass Grown on Lignocellulose Hydrolysate; Batch 2

Lignocellulose hydrolysate i.e. hemicellulose hydrolysate was prepared from wheat straw by thermo-chemical processing. After this the hydrolysate was evaporated. Before cultivation the evaporated hydrolysate was treated with activated charcoal to remove impurities and hydrolysed enzymatically to monomers.

After activated charcoal processing the hydrolysate had 3.4 w-% of phenolic compounds from the whole dry matter.

Filamentous fungus Aspergillus oryzae, strain DSM 1864 (or other A. oryzae strain which are readily available from recognized microbial culture collections) was grown under aeration in a 5-liter fermentor in fed-batch mode. Total fermentation time was 142 hours. Growth media (hemicellulose hydrolysate prepared as described above) was supplemented with yeast extract, (NH4)2SO4 (1.5 g/l), MgSO4 (1 g/l), KH2PO4 (1 g/l), K2HPO4 (2 g/l) and CaCl2 (0.1 g/l).

After cultivation biomass was inactivated by heat, harvested by filtration washed using tap-water and freeze-dried. Dried biomass was pulverized by milling and oil extracted using n-heptane. Residual solvent was removed by drying the biomass by efficient ventilation.

The dried biomass was used in pathogen binding tests.

### Preparation of Aspergillus and Mortierella Biomasses Grown on Pure Saccharides:

In a similar manner Aspergillus oryzae strain DSM 1864 biomasses were grown on glucose in a 1200-l fermenter. The growth media was supplemented with Yeast Extract (10 g/L), (NH4)2SO4 (2.5 g/L), MgCl2×6H2O (1.78 g/L), K2HPO4 (1 g/L), KH2PO4 (2 g/L), CaCl2×2H2O (0.6 g/L), ZnSO4×7H2O (0.0003 g/L), CuCl×2H2O (0.0002 g/L), MnCl2×4H2O (0.0125 (g/L).

After cultivation biomasses were inactivated by heat, harvested by filtration, washed and dried. Dried biomass was mechanically disrupted by extrusion and oil extracted using n-hexane. The solvent was removed by heating the biomass to 50° C.

In a similar manner Mortierella isabellina strain DSM 1414 (or other M. isabellina strain which are readily available from recognized microbial culture collections) biomasses were grown on glucose in a 1200-l fermenter. The growth media was supplemented with Yeast Extract (10 g/L), (NH4)2SO4 (2.5 g/L), MgCl2×6H2O (1.78 g/L), K2HPO4 (1 g/L), KH2PO4 (2 g/L), CaCl2×2H2O (0.6 g/L), ZnSO4×7H2O (0.0003 g/L), CuCl×2H2O (0.0002 g/L), MnCl2×4H2O (0.0125 (g/L)I).

After cultivation biomasses were inactivated by heat, harvested by filtration, washed and dried. Dried biomass was mechanically disrupted by extrusion and oil extracted using n-hexane. The solvent was removed by heating the biomass to 50° C.

These dried biomasses were used in pathogen binding tests.

### Preparation of Rhodosporidium Biomass Grown on Lignocellulose Hydrolysate:

Lignocellulose hydrolysate was prepared from wheat straw by thermo-chemical and enzymatic processing. After this the hydrolysate was evaporated.

Rhodosporidium toruloides strain CBS 8587 (or other R. toruloides strain, which are readily available from recognized microbial culture collections) was grown under aeration in a 10-liter fermentor. Fermentation was done as fed-batch fermentation using lignocellulosic hydrolysate syrup as the carbon source. After 10 h batch fermentation lignocellulose hydrolysate syrup was added to the fermentor periodically during the 95 h cultivation. Growth medium was supplemented with yeast extract (8 g/l), (NH4)2SO4 (2.5 g/l), MgSO4 (2.5 g/l), KH2PO4 (3.5 g/l), K2HPO4 (1.5 g/l) and CaC12 (0.1 g/l) and trace minerals ZnSO4 (0.0008 g/l), CuCl (0.00008 g/l), MnSO4 (0.0008 g/l), FeSO4 (0.0004 g/l) and NaH2PO4 (0.5 g/l).

After cultivation biomass was inactivated by heating, harvested by centrifugation washed using tap-water and dried in oven. Dried biomass was pulverized by milling and oil extracted using n-heptane. Residual solvent was removed by drying the biomass by efficient ventilation.

The dried biomass was used in pathogen binding tests.

### Preparation of Rhodosporidium Biomass Grown on Pure Saccharides:

Rhodosporidium toruloides strain CBS 8587 (or other R. toruloides strain, which are readily available from recognized microbial culture collections) was grown under aeration in a pilot-scale fermentor. Fermentation was done as fed-batch fermentation using glucose as carbon source. After 24 h batch phase glucose syrup was added to the fermentor periodically during the 143 h cultivation. Growth medium was supplemented with yeast extract (8 g/l), (NH4)2SO4 (3 g/l), MgCl2 (2 g/l), K2HPO4 (9 g/l) and CaCl2 (0.4 g/l) and trace minerals ZnSO4 (0.0003 g/l), CuCl (0.0002 g/l) and MnCl2 (0.03 g/l).

After cultivation biomass was inactivated by heat, harvested by centrifugation, washed and dried. Dried biomass was pulverized by milling and oil extracted using n-heptane. The solvent was removed by heating the biomass to 50° C. The dried biomass was used in pathogen binding tests.

### Example 2

### Pathogen Binding Tests

Tests to determine the effects of microbial biomasses as inhibitors of intestinal pathogen binding were carried out in the following way:

Microtitre plates were coated with authentic intestinal mucus from piglets. Unbound mucus was washed away with Hepes-Hanks buffer. Test products (test biomasses) were introduced into mucus coated microtiter wells. Radioactively labeled pathogen strains of E. coli (F4+) and S. enterica (serotype Dublin) in Hepes-Hanks buffer were introduced into the wells. After one-hour incubation at 37° C. loose bacteria were washed off with Hepes-Hanks buffer, scintillation liquid was added and the remaining radioactivity was measured. The remaining radioactivity was proportional to the number of adhered pathogenic bacteria. The number of pathogens bound in the wells with no potential adherence inhibitor (without biomass) (negative control) was compared to those with the test products.

The following six test products (test biomasses) were tested at four doses of biomasses:

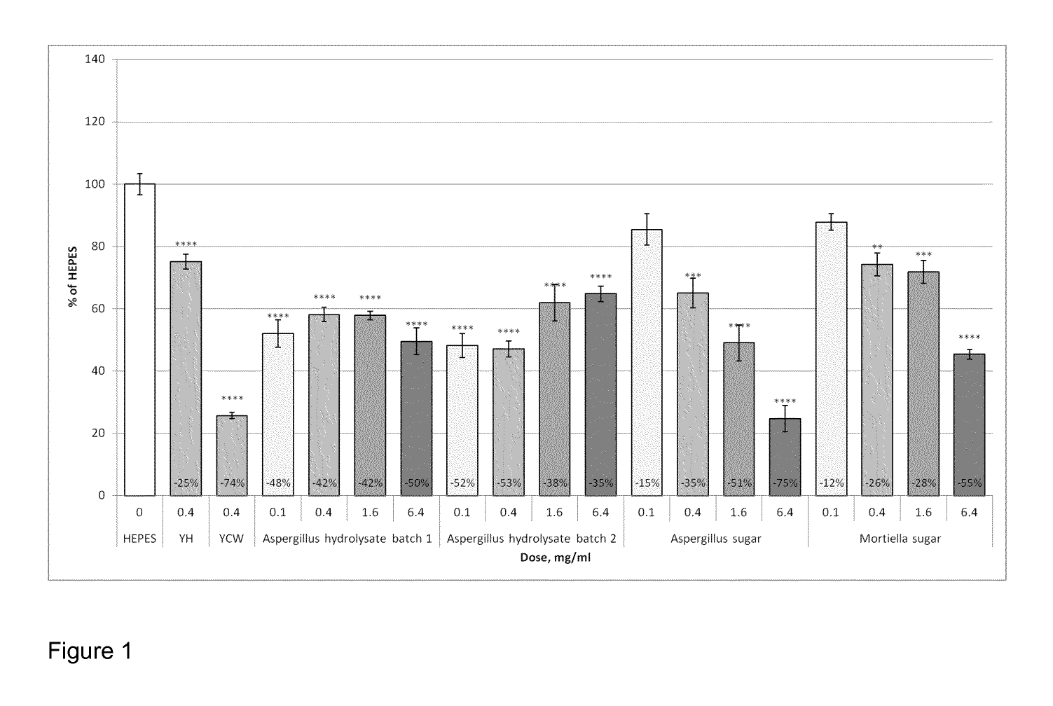

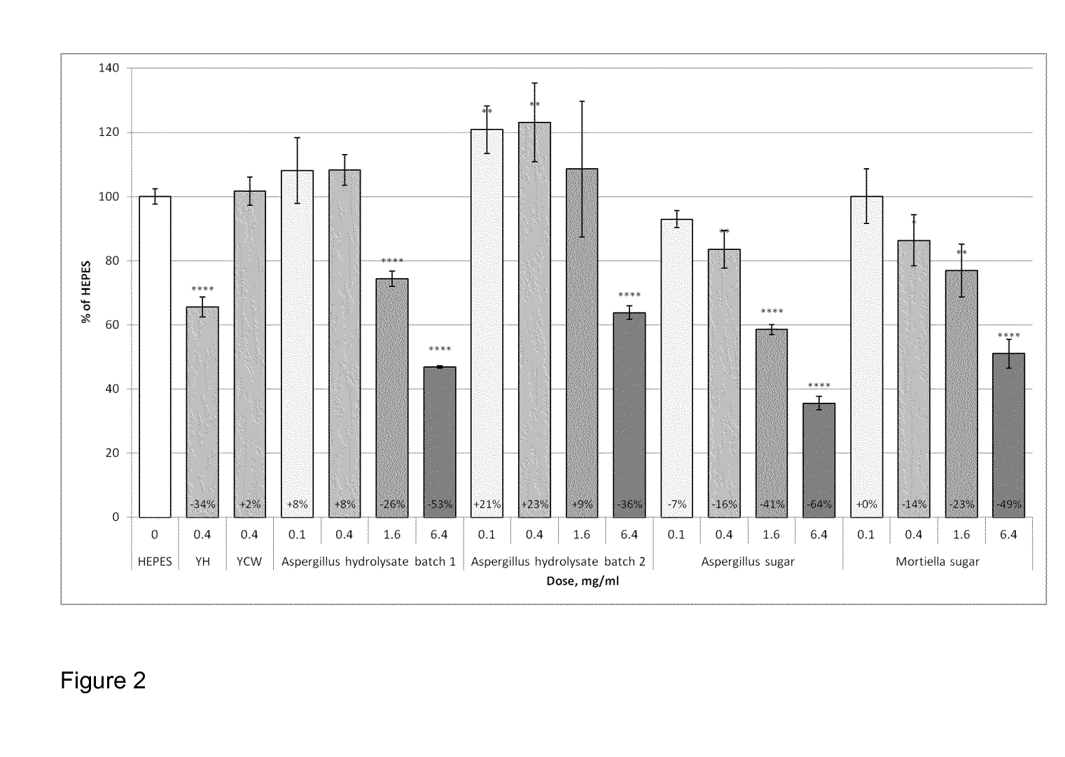

Residual microbial biomass from Aspergillus grown on lignocellulose hydrolysate, (In FIGS. 1 and 2 Aspergillus hydrolysate) batch 1

Residual microbial biomass from Aspergillus grown on lignocellulose hydrolysate, (In FIGS. 1 and 2 Aspergillus hydrolysate) batch 2

Residual microbial biomass from Aspergillus grown on saccharides (In FIGS. 1 and 2 Aspergillus sugar).

Residual microbial biomass from Mortierella grown on saccharides (In FIGS. 1 and 2 Mortierella sugar).

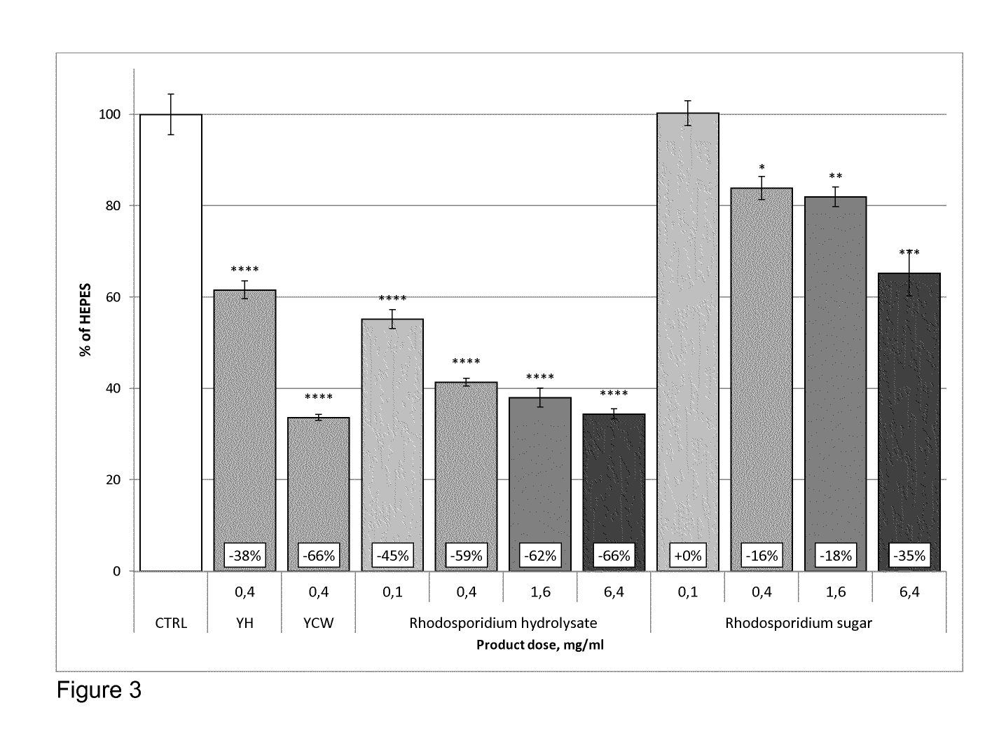

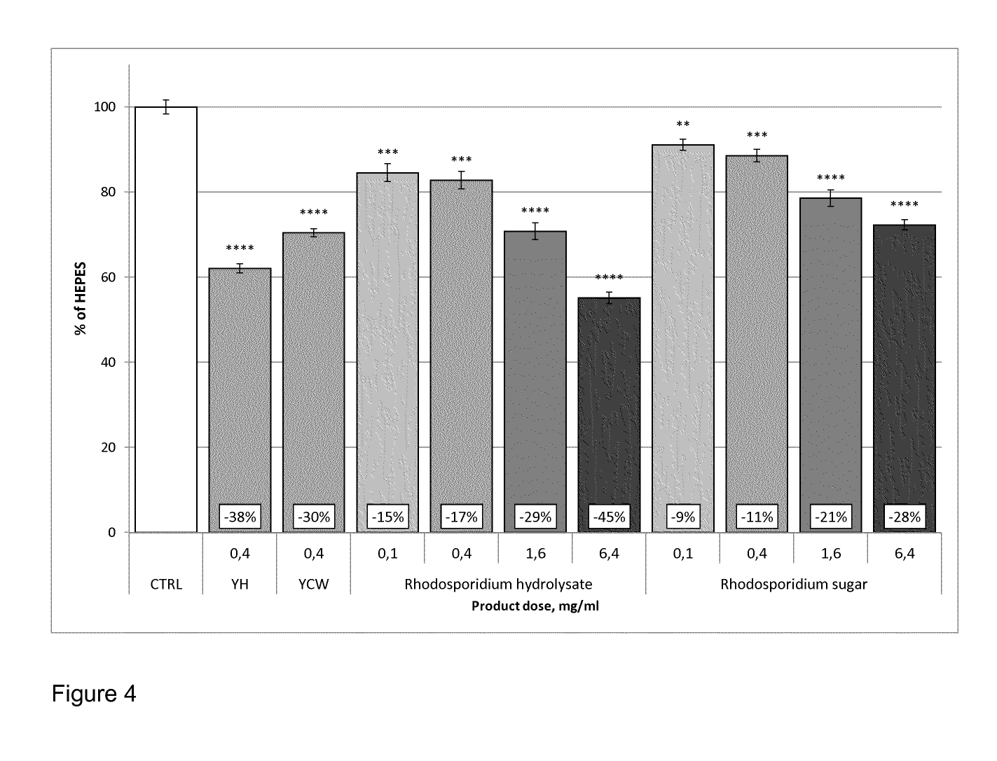

Residual microbial biomass from Rhodosporidium grown on lignocellulose hydrolysate (In FIGS. 3 and 4 Rhodosporidium hydrolysate).

Residual microbial biomass from Rhodosporidium grown on saccharides (In FIGS. 3 and 4 Rhodosporidium sugar).

Yeast biomass hydrolysate grown on saccharides was used as positive control at 0.4 mg/ml dose (In FIGS. 1, 2, 3 and 4 YH).

Also yeast cell wall (yeast grown on saccharides) was used as positive control at 0.4 mg/ml dose (In FIGS. 1, 2, 3 and 4 YCW).

No amendment was used as a negative control.

### Test Treatments

Six test product doses were used in the model, 0.1, 0.4, 1.6 and 6.4 mg/ml. Experiments were done by using four replicates.

### Effect of Fungal Biomass on the Binding of E. Coli F4+(K88) on Piglet Intestinal Epithelium.

The identities of test products and doses are shown in FIGS. 1 and 3 below the patterns of columns. Hundred % adherence of pathogens is in the absence of any test products (HEPES-Hanks buffer). Error bars indicate SE between 4 replicate reaction vessels and the number in the base of the column indicates the percentage difference when compared to HEPES. The asterisks in FIGS. 1 and 3 show the statistical significance of the difference to control with no test product according to the Student's t-test (p-value <0.05*, p-value <0.01**, p-value <0.001***, p-value <0.0001****).

Effect of Fungal Biomass on the Binding of S. enterica Serovar Dublin on Piglet Intestinal Epithelium.

The identities of test products and doses are shown in FIGS. 2 and 4 below the patterns of columns. Hundred % adherence is in the absence of any test products (HEPES-Hanks buffer). Error bars indicate SE between 4 replicate reaction vessels and the number in the base of the column indicates the percentage difference when compared to HEPES. The asterisks in FIGS. 2 and 4 show the statistical significance of the difference to control with no test product according to the Student's t-test (p-value <0.05*, p-value <0.01**, p-value <0.001***, p-value <0.0001****).

Residual microbial biomass from Aspergillus, Mortierella and Rhodosporidium grown on saccharides had similar inhibition patterns both for E. coli and S. enterica

Residual microbial biomasses from Aspergillus and Rhodosporidium grown on lignocellulose hydrolysates specifically inhibited E. coli binding even at low dose more efficiently compared to the residual microbial biomasses from Aspergillus, Mortierella or Rhodosporidium grown on saccharides (similar to the yeast cell wall product used as positive control).

Effective components in yeast are unknown, but it is hypothesized that carbohydrate components in yeast mask pathogen binding receptors on the intestinal epithelium.

Effective components in residual microbial biomasses from filamentous fungi or yeasts grown on lignocellulose are unknown. It is hypothesized carbohydrate components in microbial biomasses mask pathogen binding receptors on the intestinal epithelium and in addition compounds originating from lignocellulose hydrolysates may have additional impacts on decreasing pathogen binding to intestinal epithelium.

Results indicate that residual microbial biomasses from fungi and yeast can inhibit E. coli and S. enterica binding to intestinal epithelium. Results indicate that residual microbial biomasses from fungi and yeast grown on lignocellulose hydrolysates inhibited E. coli binding to intestinal epithelum at lower dose than residual microbial biomass grown on saccharides.

1. Microorganism biomass for use in preventing or reducing the adverse effects of pathogens in animal or human digestive tract, characterized in that said biomass comprises non-living microorganism biomass, obtainable by cultivating microorganisms on a cultivation medium comprising lignocellulosic material.

2. The biomass for use in preventing or reducing the adverse effects of pathogens in animal or human digestive tract according to claim 1, characterized in that the cultivation medium comprises lignocellulose hydrolysate.

3. The biomass for use in preventing or reducing the adverse effects of pathogens in animal or human digestive tract according to claim 1, characterized in that the cultivation medium comprises saccharides, such as mono-, di-, and/or oligosaccharides originating from lignocellulosic material.

4. The biomass for use in preventing or reducing the adverse effects of pathogens in animal or human digestive tract according to claim 1, characterized in that the microorganism biomass comprises residues of lignocellulose.

5. The biomass for use in preventing or reducing the adverse effects of pathogens in animal or human digestive tract according to claim 1, characterized that the microorganism biomass is residual microbial biomass from a single cell oil production process.

6. The biomass for use in preventing or reducing the adverse effects of pathogens in animal or human digestive tract according to claim 1, characterized that the microorganism biomass is obtainable by cultivating fungi strains, preferably yeasts or filamentous fungi.

7. The biomass for use in preventing or reducing the adverse effects of pathogens in animal or human digestive tract according to claim 1, characterized that the microorganism biomass is obtainable by cultivating filamentous fungi strains, preferably strains of Aspergillus and/or Mortierella genus or yeasts preferably strains of Rhodosporidium or Lipomyces genus.

8. The biomass for use in preventing or reducing the adverse effects of pathogens in animal or human digestive tract according to claim 1, characterized in the microorganism biomass prevents or reduces the adverse effects of pathogens in animal or human stomach, small intestine and/or colon.

9. The biomass for use in preventing or reducing the adverse effects of pathogens in animal or human digestive tract according to claim 1, characterized in that said microorganism biomass prevents or reduces the adverse effects of pathogens comprising Salmonella genera and E. coli.

10. The biomass for use in preventing or reducing the adverse effects of pathogens in animal or human digestive tract according to claim 9, characterized in that said microorganism biomass prevents or reduces the adverse effects of pathogens Escherichia coli and/or Salmonella enterica.

11. A feed or food composition for use in preventing or reducing the adverse effects of pathogens in animal or human digestive tract, characterized in that it comprises the microorganism biomass according to 1.

12. The composition for use in preventing or reducing the adverse effects of pathogens in animal or human digestive tract according to claim 11, characterized in that the composition comprises the microorganism biomass 0.001-20 wt %, preferably 0.1-3 wt % dry weight of dry weight of said composition.

13. The composition for use in preventing or reducing the adverse effects of pathogens in animal or human digestive tract according to claim 11, characterized in that the composition is a ruminant or monogastric animal feed composition.

14. The composition for use in preventing or reducing the adverse effects of pathogens in animal or human digestive tract according to claim 11, characterized in that the food composition is a nutritional product for human use.

15. The composition for use in preventing or reducing the adverse effects of pathogens in animal or human digestive tract according to claim 11, characterized in that said microorganism biomass is used in the composition as a food or feed topping, food or feed extender or feed or food supplement.

16. A feed or food composition, characterized in that the composition comprises 0.001-20 wt % dry weight of dry weight of said composition non-living microorganism biomass as defined in claim 1.

17. The composition according to claim 16, characterized in that the composition comprises 0.1-3 wt % dry weight of dry weight of said composition microorganism biomass.

18. A method for improving the well-being and increasing the productivity of an animal, characterized in that said method comprises feeding to said animal a feed composition comprising non-living microorganism biomass as defined in claim 1.

19. The method according to claim 18, characterized in that the composition comprises 0.001-20 wt %, preferably 0.1-3 wt % dry weight of dry weight of said composition microorganism biomass.

20. A method for preparing a feed additive, said method comprising the steps of
(a) cultivating one or more microorganism(s) in a cultivation medium comprising lignocellulosic material,
(b) subject said microorganism(s) to a step of cell disruption to obtain non-living biomass of said microorganism(s)
(c) isolate a solid phase containing the microbial biomass and residues of lignocellulose of step (b),
(d) optionally, drying the microbial biomass obtained from step (c).

21. The method of claim 20, wherein the microbial biomass obtained from (c) is subjected to washing step.

22. The method according to claim 20, wherein said cultivation medium comprising lignocellulose hydrolysate.

23. The method of according to claim 20, wherein said cultivation medium comprises starch and/or sugar cane/beet derived sugars.

24. The method of according to claim 20, wherein the microbial biomass is obtained from oleaginous microorganisms.

25. The method of according to claim 20, wherein said microbial biomass is residual microbial biomass obtained from a fermentation process.

26. The method of according to claim 24, wherein the microbial biomass of step (c) is obtained by subjecting the oleaginous microorganisms to a step of removing the microbial oil from said microbial biomass.

27. The method of according to claim 26, wherein the microbial biomass of step (c) is obtained by subjecting the oleaginous microorganisms to a step of removing the microbial oil from said microbial biomass after step b).

28. The method of according to claim 20, wherein the microbial biomass is obtained from one or more microorganism.

29. The method of according to claim 20, wherein said microbial biomass is obtained from one or more fungi, preferably yeasts or filamentous fungi.

30. The method of according to claim 20, wherein said microbial biomass is obtained from Rhodosporidium.

31. The method of according to claim 20, wherein said microbial biomass is obtained from Aspergillus or Mortierella.

32. A feed or food additive obtainable from the method of claim 20.

33. A method for inactivating a pathogen in a contaminated feed or food product, said method comprising the steps of
(a) providing a feed or food product contaminated with a pathogen,
(b) providing a feed or food additive according to claim 32,
(c) mixing the feed or food product of (a) with the feed or food additive of (b).

34. A method for preparing a feed or food product, said method comprising the steps of
(a) providing a feed or food substance,
(b) providing a feed or food additive according to claim 32,
(c) mixing the feed or food substance of (a) with the feed or food additive of (b) to obtain a feed or food product.

35. The method according to claim 33, wherein the feed additive amounts 0.001-20 wt % dry weight of the feed of step (c).

36. The method according to claim 33, wherein the feed additive amounts 0.1-3 wt % dry weight of the feed of step (c).

37. The method according to claim 33, wherein the feed or food product is contaminated with a pathogen selected from the list consisting of Salmonella genera and E. coli or a combination of said pathogens.

38. The method according to claim 33, wherein the feed or food product is contaminated with a pathogen selected from the list consisting of Escherichia coli and Salmonella enterica or a combination of said pathogens.

39. A feed or food product obtainable of claim 34.

40. A method for feeding an animal, said method comprising
(a) feeding said animal with a feed product according to claim 39.

41. The method according to claim 40, wherein said animal is a ruminant or monogastric animal.

42. The method according to claim 40, wherein said animal is aquaculture.

43. The method according to claim 40, wherein the animal is a livestock.

44. The method according to claim 40, wherein said animal is a livestock selected from the list consisting of pigs, horses, poultry, cattle, goats and sheep.

45. The method according to claim 40, wherein said animal is a chicken.

46. The method according to claim 40, wherein said animal is a pig.
